(19) United States
(12) Patent Application Publication
(10) Pub. No.: US 2020/0253257 A1
(43) Pub. Date: Aug. 13, 2020

(54) SUGAR REPLACEMENT COMPOSITION AND METHOD OF USING THE SAME

(71) Applicant: T-BEV, INC., Eugene, OR (US)

(72) Inventor: Yihai Jiang, Irvine, CA (US)

(21) Appl. No.: 16/786,924

(22) Filed: Feb. 10, 2020

Related U.S. Application Data
(60) Provisional application No. 62/802,959, filed on Feb. 8, 2019.

(51) Int. Cl. A23L 33/125; A23L 27/30; A61K 8/60; A61Q 11/00

(52) U.S. Cl. CPC A23L 33/125; A23L 27/33; A23V 2002/00; A61Q 11/00; A61K 8/60

(57) ABSTRACT

A sweetening composition includes a mixture of isomaltulose as a major component and a sweetener as a minor component, wherein the sweetener is one or more selected from the group consisting of stevia, luo han guo (monk fruit), pentadin, brazzein, monellin, thaumatin, sucralose, aspartame, acesulfame potassium, saccharin, neotame, and advantame, and wherein the sweetening composition has a relative sweetness which is the same as regular sugar.

### CROSS-REFERENCE TO RELATED APPLICATION(S)

This utility patent application claims priority to and the benefit of U.S. Provisional Patent Application Ser. No. 62/802,959, filed Feb. 8, 2019, entitled “SUGAR REPLACEMENT COMPOSITIONS WITH HEALTH BENEFITS,” the entire content of which is incorporated herein by reference.

### FIELD

One or more aspects of example embodiments of the present invention relate to sugar replacement composition and method of utilizing the same.

### BACKGROUND

Sugar is utilized broadly in food, beverages, diet supplements, medications and may other consumable or injectable products. While sugar provides the desirable sweetness, it has also been linked to health issues, such as obesity, diabetes, etc.

There remains a need for a sugar replacement composition that reduces the unwanted side effects of sugar.

### SUMMARY

According to an embodiment, a sweetening composition consists essentially of a mixture of isomaltulose as a major component and a sweetener as a minor component, wherein the sweetener is one or more selected from the group consisting of stevia, luo han guo (monk fruit), pentadin, brazzein, monellin, thaumatin, sucralose, aspartame, acesulfame potassium, saccharin, neotame, and advantame, and wherein the sweetening composition has a relative sweetness which is the same as regular sugar.

In one embodiment, the sweetener is 0.33 wt % or less in amount based on a total weight of the sweetening composition.

In one embodiment, the sweetener is 0.2 wt % in amount based on a total weight of the sweetening composition.

In one embodiment, an amount of calories per unit weight of the sweetening composition is the same as an amount of calories per unit weight of regular sugar.

According to an embodiment, an ingestible composition includes the sweetening composition.

In one embodiment, the ingestible composition has low glycemic effects and is in a form of a beverage.

According to an embodiment, an oral product includes the sweetening composition.

According to an embodiment, a method for sweetening ingestible compositions and/or oral products includes incorporating the sweetening composition in the ingestible compositions and/or oral products.

In an embodiment, the oral product is one or more selected from the group consisting of mouthwash and toothpaste.

In an embodiment, the ingestible composition is one or more selected from the group consisting of foodstuffs, medicaments, nutritional supplement products, flavored confectionery tablets, powder drink mixes and beverages.

According to an embodiment, a method for supporting weight loss and/or management includes replacing sugar in an ingestible composition and/or oral product with an equal amount of the sweetening composition.

According to an embodiment, a method for improving athletic performance includes replacing sugar in an ingestible composition and/or oral product with an equal amount of the sweetening composition.

According to an embodiment, a method for improving cognitive performance includes replacing sugar in an ingestible composition and/or oral product with an equal amount of the sweetening composition.

According to an embodiment, a method for improving dental health includes replacing sugar in an ingestible composition and/or oral product with an equal amount of the sweetening composition.

According to an embodiment, a method for supporting prevention and/or management of diabetes mellitus includes replacing sugar in an ingestible composition and/or oral product with an equal amount of the sweetening composition.

The method may further include utilizing the ingestible composition and/or oral product by a consumer.

### DETAILED DESCRIPTION

Hereinafter, example embodiments will be described in more detail. The terminology used herein is for the purpose of describing particular embodiments and is not intended to be limiting of the present invention. As used herein, the singular forms “a” and “an” are intended to include the plural forms as well, unless the context clearly indicates otherwise. It will be further understood that the terms “comprises,” “comprising,” “includes,” and “including,” when used in this specification, specify the presence of the stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless otherwise defined, all terms (including technical and scientific terms) used herein have the same meaning as commonly understood by one of ordinary skill in the art to which the present invention belongs. It will be further understood that terms, such as those defined in commonly used dictionaries, should be interpreted as having a meaning that is consistent with their meaning in the context of the relevant art and/or the present specification, and should not be interpreted in an idealized or overly formal sense, unless expressly so defined herein.

Hereinafter, the term “sugar replacement composition” and the term “sweetening composition” are used interchangeably.

According to an embodiment of the present disclosure, a sweetening composition includes a mixture of isomaltulose and one or more sweeteners.

Isomaltulose is a disaccharide carbohydrate composed of glucose and fructose. The glucose and fructose are linked by an alpha-1,6-glycosidic bond (chemical name: 6-O-α-D-Glucopyranosyl-D-fructose). Sweeteners are substances added to foodstuff and other related items in order to make them sweet.

The sweeting composition includes isomaltulose as a major component and the one or more sweeteners as a minor component. That is, isomaltulose is included at a much greater amount than the one or more sweeteners.

The sweeteners in the sweetening composition may be natural and/or artificial, and may be selected from the group consisting of stevia, luo han guo (monk fruit), pentadin, brazzein, monellin, thaumatin, sucralose, aspartame, acesulfame potassium, saccharin, neotame, and advantame.

The relative quantity (e.g., weight ratio) of the isomaltulose and the sweetener is selected such as to provide a relative sweetness which is substantially the same (e.g., the same) as regular sugar.

Throughout this disclosure, the term “regular sugar” refers to table sugar, or sucrose.

Throughout this disclosure, the term “relative sweetness” refers to the taste of sweetness provided by a material, e.g., the sweetening composition according to embodiments of the present disclosure, relative to the taste of sweetness provided by regular sugar of the same weight. In other words, relative sweetness is the material's sweetness compared to sucrose. The relative sweetness of some common nutritive sweeteners are listed in Table 1.

TABLE 1

| | Relative |
|---|---|
| | Sweetness |
| NUTRITIVE SWEETENERS | (sucrose = 1) |
| Agave syrup (56-92% fructose + glucose + water) | 1.4 |
| Fructose (fruit sugar) | 1.2-1.8 |
| Galactose | 0.3 |
| Glucose (dextrose, grape sugar) | 0.7 |
| High fructose corn syrup HFCS-42 (32% fructose + 40% | 0.9 |
| glucose + other sugars + 29% water) | |
| High fructose corn syrup HFSC-55 (42% fructose + 32% | 1.1 |
| glucose + other sugars + 23% water) | |
| Isomaltulose | 0.4 |
| Lactose (milk sugar) | 0.2 |
| Maple syrup (66% sucrose + other sugars + 33% water) | 0.6 |
| Sucrose (table sugar) | 1 |

Throughout this disclosure, the term “sweetness,” and “taste of sweetness” are used interchangeably. It refers to the sweetness perceived by a consumer, or measured by a suitable method.

Throughout this disclosure, the expression “a relative sweetness which is the same as regular sugar” refers to that when the sweetening composition of the present disclosure is utilized at a given weight in a formulation, it will provide the same sweetness as when regular sugar of the same weight is utilized. That is, the sweetening composition of the present disclosure has a relative sweetness of about 1 and can be utilized to replace regular sugar at the same amount as required by a given recipe or formulation and provides the same sweetness as if regular sugar itself was utilized.

For example, 20 grams of the sweetening composition may be utilized to replace 20 grams of white cane sugar in making a foodstuff, and provide the same sweetness as the 20 grams of granulated white cane sugar.

The amount of the sweetener in the sweetening composition according to embodiments of the present disclosure is very low. In one embodiment, the amount (e.g., weight percent) of the sweetener in the sweetening composition is 0.33 wt % or less based on the total weight of the sweetening composition, and the sweetness of the sweetening composition is the same as regular sugar. In one embodiment, the amount of the sweetener in the sweetening composition is 0.2 wt % or less. In one embodiment, the amount of the sweetener in the sweetening composition is from 0.01 wt % or more to 0.33 wt % or less.

Even though the sweetener is included at a very low amount in the sweetening composition, the mixture of isomaltulose and one or more sweeteners has the synergistic effect of providing a relative sweetness which is substantially the same (e.g., the same) as regular sugar.

In one embodiment, the sweetening composition includes only isomaltulose and one or more sweeteners.

The sweetening composition may be utilized for sweetening ingestible compositions and/or oral products.

In one embodiment, the sweetener in the sweetening composition may include an artificial sweetener selected from sucralose, aspartame, acesulfame potassium, saccharin, neotame, and advantame.

In one embodiment, the sweetener in the sweetening composition may include a natural sweetener selected from stevia, luo han guo (monk fruit), pentadin, brazzein, monellin, and thaumatin.

In one embodiment, the sweeteners in the sweetening composition are stevia, sucralose, aspartame, and/or acesulfame potassium.

In one embodiment, the sweetening composition may provide similar (e.g., about the same) amount of calories compared to regular sugar, e.g., per unit weight. That is, when a given amount of the sweetening composition is consumed, it will generate the same amount of calories as the same amount of sugar.

Ingestion of the invented sweetening composition may result in lower blood glucose and lower insulin response compared to the regular sugar. The invented sweetening composition has a low glycemic index (GI). For example, the invented sweetening composition may have a lower glycemic index than regular sugar.

Without being bound by any particular theories, this may be because Isomaltulose in the invented sweetening composition is slow to be digested and absorbed, and therefore it is released as glucose and fructose into the bloodstream gradually (e.g., slowly). Consequently, the invented sweetening composition has a low glycemic index (GI). This will support the prevention and management of diabetes mellitus, cardiovascular disease, and/or possibly overweight.

Ingestion of the invented sweetening composition has been associated with higher rates of fat oxidation and lower rates of fat storage compared to regular sugar. Since the invented sweetening composition has a lower blood glucose concentration, which then provides a lower stimulus to insulin secretion, which in turn allows more fatty acids to be released from adipose tissue for oxidation as an energy source. This will be helpful in weight loss and management.

Ingestion of the invented sweetening composition has showed slow and sustained release of glucose during exercise compared to regular sugar. This results in improved athletic performance.

Ingestion of the invented sweetening composition has shown to improve cognitive performance, e.g., mood and memory, as compared to regular sugar.

The invented sweetening composition is good for dental health by resisting fermentation by oral bacteria.

The invented sweetening composition may be utilized to replace sugar (e.g., white granulated cane sugar) at a ratio of 1:1 in ingestible compositions and oral products. Such formulated sweetening composition with health benefits will make it easier to replace regular sugar in all existing ingestible compositions and oral products in the market.

According to an embodiment of the present disclosure, an ingestible composition includes the sweetening composition described above.

The ingestible composition may have low glycemic effects and may be in the form of a beverage.

According to an embodiment of the present disclosure, an oral product includes the sweetening composition described above. The oral product has low glycemic effects.

According to an embodiment of the present disclosure, a method of sweetening an ingestible compositions and/or oral products includes incorporating therein the sweetening composition described above. The sweetening composition may be added at the same or substantially same amount as the regular sugar required in the recipe or formulation.

The oral product may be one or more selected from the group consisting of chewing gum, mouthwash and toothpaste.

The ingestible composition may be selected from the group consisting of foodstuff, medicament, nutritional supplement products, flavored confectionery tablet, powder drink mixes and beverage.

According to an embodiment of the present disclosure, a method of supporting prevention and management of diabetes mellitus includes replacing sugar in an ingestible composition or oral product with an equal amount of the sweetening composition described above. The method may further include consuming or utilizing the ingestible composition and/or oral product by a consumer.

According to an embodiment of the present disclosure, a method of supporting weight loss and management includes replacing sugar in an ingestible composition or oral product with an equal amount of the sweetening composition described above. The method may further include consuming or utilizing the ingestible composition and/or oral product by a consumer.

According to an embodiment of the present disclosure, a method of improving athletic performance includes replacing sugar in an ingestible composition or oral product with an equal amount of the sweetening composition described above. The method may further include utilizing of the ingestible composition and/or oral product by a consumer.

According to an embodiment of the present disclosure, a method of improving cognitive performance, such as mood and memory, includes replacing sugar in an ingestible composition and/or oral product with an equal amount of the sweetening composition described above. The method may further include utilizing of the ingestible composition or oral product by a consumer.

According to an embodiment of the present disclosure, a method of improving dental health includes replacing sugar in an ingestible composition and/or oral product with an equal amount of the sweetening composition described above. The method may further include utilizing of the ingestible composition or oral product by a consumer.

The sweetening composition according to one or more embodiments of the present disclosure may be utilized to replace regular sugar (e.g., cane sugar) in any recipe or formulation in a 1:1 ratio. In another embodiment, the sweetening composition may be utilized to replace other type of sweetening materials, such as honey, brown sugar, artificial or natural sweeteners.

In one embodiment, no other change is needed except for replacing regular sugar with the sweetening composition according to embodiments of the present disclosure, and the above discussed health benefits may be obtained.

### EXAMPLES

### Example 1

A sweetening composition includes 99.80 wt % isomaltulose and 0.20 wt % stevia.

### Example 2

A sweetening composition includes 99.90 wt % isomaltulose and 0.10 wt % sucralose.

### Example 3

A sweetening composition includes 99.67 wt % isomaltulose and 0.33 wt % aspartame.

### Example 4

A sweetening composition includes 99.70 wt % isomaltulose and 0.30 wt % acesulfame potassium.

### Example 5

A sweetening composition includes 99.88 wt % isomaltulose and 0.12 wt % pentadin.

### Example 6

A sweetening composition includes 99.99 wt % isomaltulose and 0.01 wt % neotame.

### Example 7

A sweetening composition includes 99.94 wt % isomaltulose and 0.06 wt % brazzein.

### Example 8

A sweetening composition includes 99.96 wt % isomaltulose and 0.04 wt % monellin.

### Example 9

A sweetening composition includes 99.97 wt % isomaltulose and 0.03 wt % thaumatin.

### Example 10

A sweetening composition includes 99.85 wt % isomaltulose, 0.05 wt % sucralose and 0.1 wt % stevia.

The replacing of sugar in the ingestible composition or oral product with an equal amount of the sweetening composition described above allows the use of the same recipe for manufacturing the ingestible composition or oral product, without any additional burden on the operator in changing the amount or ratio of each of the ingredients. Further, the manufacturing can be conducted on the same equipment as when sugar is used.

The replacing of sugar in the ingestible composition or oral product with an equal amount of the sweetening composition described above results in approximately no change or no change at all in the sweetness of the ingestible composition or oral product, thereby making no change on the taste of the ingestible composition or oral product. Therefore, the consumer may detect no change in the respective product they use, but may gain the health benefits described above.

In this specification and in the claims, the term “ingestible composition” is used to mean a composition which is intended to be taken into the mouth and swallowed, substantially in its entirety. Examples include foodstuffs, beverages, medicaments, nutritional supplement products and confectionery as well as dry mixes and concentrates for use in the preparation of such compositions. The term “oral product” is used to mean a substance which is intended to be taken into the mouth but not swallowed. Examples include toothpastes, mouthwashes and other buccal and dental preparations.

The sweetening composition according to embodiments of the present disclosure has the unexpected synergistic results of having substantially the same relative sweetness and calories as regular sugar.

Expressions such as “at least one of” or “at least one selected from” when preceding a list of elements, modify the entire list of elements and do not modify the individual elements of the list. Further, the use of “may” when describing embodiments of the present invention refers to “one or more embodiments of the present invention.” Also, the term “exemplary” is intended to refer to an example or illustration.

As utilized herein, the term “substantially,” “about,” and similar terms are utilized as terms of approximation and not as terms of degree, and are intended to account for the inherent deviations in measured or calculated values that would be recognized by those of ordinary skill in the art. Moreover, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein. All such ranges are intended to be inherently described in this specification such that amending to expressly recite any such subranges would comply with the requirements of 35 U.S.C. § 112, first paragraph, or 35 U.S.C. § 112(a), and 35 U.S.C. § 132(a).

While this invention has been described in detail with particular references to illustrative embodiments thereof, the embodiments described herein are not intended to be exhaustive or to limit the scope of the invention to the exact forms disclosed. Persons skilled in the art and technology to which this invention pertains will appreciate that alterations and changes in the described structures and methods of assembly and operation can be practiced without meaningfully departing from the principles, spirit, and scope of this invention, as set forth in the following claims and equivalents thereof.

What is claimed is:

1. A sweetening composition consisting essentially of a mixture of isomaltulose as a major component and a sweetener as a minor component,
wherein the sweetener is one or more selected from the group consisting of stevia, luo han guo (monk fruit), pentadin, brazzein, monellin, thaumatin, sucralose, aspartame, acesulfame potassium, saccharin, neotame, and advantame, and
wherein the sweetening composition has a relative sweetness which is the same as regular sugar.

2. The sweetening composition of claim 1, wherein the sweetener is 0.33 wt % or less in amount based on a total weight of the sweetening composition.

3. The sweetening composition of claim 1, wherein the sweetener is 0.2 wt % in amount based on a total weight of the sweetening composition.

4. The sweetening composition of claim 1, wherein an amount of calories per unit weight of the sweetening composition is the same as an amount of calories per unit weight of regular sugar.

5. An ingestible composition comprising the sweetening composition of claim 1.

6. The ingestible composition of claim 5, wherein the ingestible composition has low glycemic effects and is in a form of a beverage.

7. An oral product comprising the sweetening composition of claim 1.

8. A method for sweetening ingestible compositions and/or oral products, the method comprising incorporating the sweetening composition of claim 1 in the ingestible compositions and/or oral products.

9. The method of claim 8, wherein the oral product is one or more selected from the group consisting of mouthwash and toothpaste.

10. The method of claim 8, wherein the ingestible composition is one or more selected from the group consisting of foodstuffs, medicaments, nutritional supplement products, flavored confectionery tablets, powder drink mixes and beverages.

11. A method for supporting weight loss and management, the method comprising replacing sugar in an ingestible composition and/or oral product with an equal amount of the sweetening composition of claim 1.

12. A method for improving athletic performance, the method comprising replacing sugar in an ingestible composition and/or oral product with an equal amount of the sweetening composition of claim 1.

13. A method for improving cognitive performance, the method comprising replacing sugar in an ingestible composition and/or oral product with an equal amount of the sweetening composition of claim 1.

14. A method for improving dental health, the method comprising replacing sugar in an ingestible composition and/or oral product with an equal amount of the sweetening composition of claim 1.

15. A method for supporting prevention and/or management of diabetes mellitus, the method comprising replacing sugar in an ingestible composition and/or oral product with an equal amount of the sweetening composition of claim 1.

16. The method of claim 15, further comprising utilizing the ingestible composition or oral product by a consumer.
